(19) United States
(12) Patent Application Publication
(10) Pub. No.: US 2016/0123943 A1
(43) Pub. Date: May 5, 2016

(54) GAS RECOGNITION METHOD BASED ON COMPRESSIVE SENSING THEORY

(71) Applicant: INSTITUTE OF MICROELECTRONICS, CHINESE ACADEMY OF SCIENCES, Beijing (CN)

(72) Inventors: Dongmei LI, Beijing (CN); Hao ZHANG, Beijing (CN); Shengfa LIANG, Beijing (CN); Qing LUO, Beijing (CN); Xiaojing LI, Beijing (CN); Changqing XIE, Beijing (CN); Ming LIU, Beijing (CN)

(21) Appl. No.: 14/895,915

(22) PCT Filed: Jun. 5, 2013
(86) PCT No.: PCT/CN2013/076759

§ 371 Date: Dec. 3, 2015

(51) Int. Cl. G01N 33/00

(52) U.S. Cl. CPC G01N 33/0034

(57) ABSTRACT

A gas recognition method based on a compressive sensing theory. The method comprises: collecting compressed data in an under-sampling manner; performing a reconstruction on the collected compressed data to obtain reconstructed data; training a back-propagation neural network by using the reconstructed data and storing the trained back-propagation neural network; inputting data under test into the trained back-propagation neural network, such that the trained back-propagation neural network performs a recognition on the data under test to realize qualitative recognition of gas. The method solves the problem in transmission and storage of large amount of data and the problem of imprecise recognition in current gas detection, and achieves the object that a precise qualitative recognition is achieved by using a reduced amount of data.

### CROSS-REFERENCE TO RELATED APPLICATION(S)

This application is a U.S. national phase application of PCT Application No. PCT/CN2013/076759 filed on Jun. 5, 2013, entitled “GAS RECOGNITION METHOD BASED ON COMPRESSIVE PERCEPTION THEORY”. This PCT Application is incorporated herein by reference in its entirety.

### TECHNICAL FIELD

The present disclosure relates to the field of sensor array signal processing technology, and in particular, to a gas recognition method based on the compressive sensing theory.

### BACKGROUND

Due to ubiquitous cross-sensitivity of a gas sensor, its output signal is affected by factors, such as temperature, humidity, and environmental conditions, and therefore its stability and selectivity is poor. As a result, its application is limited to scenarios where the requirement for detecting accuracy is low or components of gas are simple, such as hazardous gas leakage alarm scenario. In a conventional solution, the above adverse effects are eliminated by finding a new sensitive material, a new device structure, and a compensation circuit. However, such a solution not only complicates device structures, but also increases costs for device manufactures.

The compressive sensing theory is a brand new theory of signal collection and encoding/decoding which utilizes signal sparsity or compressibility. The basic concept of this theory is to extract information as much as possible from data as little as possible. The theory states that as long as a signal is sparse in a certain domain, the signal may be projected by using a non-correlation matrix, and then an optimization question may be solved by using a reduced amount of these projected values, and finally it is highly possible to reconstruct the original signal. In other words, a loss-less sampling of a compressible signal may be achieved at a rate which is much lower than the Nyquist sampling rate, and then the original data may be reconstructed accurately based on the reduced amount of the data.

A neural network may effectively solve the problem of non-linearity caused by cross-sensitivity of a gas sensor and suppress drift or noise of the sensor to an extent, by using its capabilities of non-linear mapping, parallel processing, and highly self-learning, self-organizing, and adaption. This facilitates improving the accuracy of gas detection. Therefore, a detection technology in which a high performance and low cost gas sensor is combined with a smart recognition technology represented by neural network is becoming a trend in gas detection nowadays.

In a neural network, a back propagation (BP) neural network is a multilayer feed-forward network trained according to “back propagation of error” algorithm. Typically, it is composed of multiple network layers comprising an input layer, one or more hidden layers, and an output layer. An interconnection is used between layers, and no interconnection is used between neurons within a same layer. Neurons in a hidden layer typically uses a Sigmoid-type transfer function, and neurons in the output layer uses a purelin-type transfer function. A training procedure of a NP neural network is composed of a forward propagation and a back propagation. During the forward propagation, an input pattern is processed by the input layer and the hidden layers one by one, and finally propagated to the output layer. If an expected output cannot be acquired at the output layer, then the back propagation procedure begins. During the back propagation, error values are propagated backward layer by layer via the interconnections, and connection weight in each layer is corrected until an expected training error is achieved.

### SUMMARY

### Problems to be Solved

In order to solve the above defects of the related art, the present disclosure provides a gas recognition method based on the compressive sensing theory to solve the problem in transmission and storage of large amount of data and the problem of imprecise recognition in current gas detection, and to achieve the object that a precise qualitative recognition is achieved by using a reduced amount of data.

### Solutions to the Problems

In order to achieve the above object, the present disclosure provides a gas recognition method based on the compressive sensing theory. The method comprises: step 1 of collecting compressed data in an under-sampling manner; step 2 of performing a reconstruction on the collected compressed data to obtain reconstructed data; step 3 of training a back-propagation neural network by using the reconstructed data and storing the trained back-propagation neural network; and step 4 of inputting data under test into the trained back-propagation neural network, such that the trained back-propagation neural network performs a recognition on the data under test to realize qualitative recognition of gas.

In the above solution, step 1 of collecting compressed data in an under-sampling manner, specifically comprises: collecting, by array nodes of a sensor network, compressible original data; performing a sparse decomposition on the original data to acquire a first sparse matrix which is a sparse matrix correlated to the original data; performing a non-linear projection processing on the first sparse matrix to acquire a second sparse matrix whose elements are random combinations of elements of the first sparse matrix; and performing a low rate under-sampling on the data of the second sparse matrix having a greater coefficient at a frequency lower than the Nyquist sampling frequency.

In the above solution, performing the sparse decomposition on the original data to acquire a first sparse matrix comprises: constructing a sparse matrix with a random Gaussian distribution and multiplying the collected original data by this constructed sparse matrix with the random Gaussian distribution to acquire the first sparse matrix. The constructing of a sparse matrix with a random Gaussian distribution comprises: selecting a Gaussian matrix with dimensions of M×N, each element of which Gaussian matrix obeying the Gaussian distribution; and then normalizing each column of the Gaussian matrix to acquire a sparse matrix ψ.

In the above solution, multiplying the collected original data by this constructed sparse matrix with the random Gaussian distribution to acquire the first sparse matrix comprises: multiplying collected compressible original data X by the constructed sparse matrix with the random Gaussian distribution ψ to acquire the first sparse matrix which is the sparsest representation of the compressible original data X, wherein if the number K of non-zero elements in the first sparse matrix is less than the number of non-zero elements in the compressible original data X, then the first sparse matrix is compressible.

In the above solution, step 2 of performing the reconstruction on the collected compressed data to obtain reconstructed data, comprises: selecting an observation matrix which is not correlated to the first sparse matrix; multiplying collected compressed data by the observation matrix to acquire the observed data; and performing an inverse transform on the observed data to acquire the reconstructed data.

In the above solution, the selected observation matrix is an observation matrix P with dimensions of M×N, and the observation matrix P is not correlated to the first sparse matrix. The collected compressed data X is multiplied by the observation matrix P, to acquire the observed data Y which is a linear combination of column vectors in the observation matrix P corresponding to the non-zero vectors in the first sparse matrix. The performing of an inverse transform on the observed data is to solve X in an equation of PψX=Y, where θ=ψX. Since the number of unknown quantities in this equation set is greater than the number of equations in the equation set, the solution of X is not unique. Here, the least-1-norm is used to approximate the solution, and the final result is the reconstructed data.

In the above solution, step 3 of training a back-propagation neural network by using the reconstructed data and storing the trained back-propagation neural network, specifically comprises: inputting the reconstructed data, as input samples, to the back propagation neural network; processing, by the back propagation neural network, the reconstructed data iteratively and comparing the iterative error in each step with that in its previous step; and stopping the training and storing the back-propagation neural network when the iterative error reaches at an initially set threshold.

In the above solution, step 4 of performing, by the trained back-propagation neural network, a recognition on the data under test is that the trained back-propagation neural network compares respective connection weights during the training and outputs binary quantized numbers with the most number of similar weights, the output binary quantized numbers representing different kinds of gases, such that a qualitative recognition of gas is achieved.

### ADVANTAGEOUS EFFECT

From the above solutions, the present disclosure has advantageous effects as follows:

1. The gas recognition method based on the compressive sensing theory provided by the present disclosure applies the compressive sensing technology to the propagation procedure of the array signals in the sensor network, and uses a BP neural network at the receiver side to train the recognition. In this way, the computation complexity is transferred from online detection to offline network training, such that the timeliness and accuracy for the online detection done by the detection system are greatly improved. This solves the problem in transmission and storage of large amount of data and the problem of imprecise recognition in current gas detection, and achieves the object that a precise qualitative recognition is achieved by using a reduced amount of data.

2. In contrast to the related art, the gas recognition method based on the compressive sensing theory provided by the present disclosure combines the compressive sensing theory and the neural network, and compresses and transmits the data simultaneously. After that, a BP neural network is used at backend to train the recognition of the compressed reconstructed data. This may effectively improve the data storage capability and bandwidth usage, and an accurate recognition may be achieved which facilitates improving accuracy of gas detection.

### BRIEF DESCRIPTION OF THE DRAWINGS

To further illustrate the content of the present disclosure, a detailed description of the present disclosure will be given in conjunction with the drawings and embodiments, in which:

FIG. 1 is a flow chart of a gas recognition method based on the compressive sensing theory according to an embodiment of the present disclosure;

FIGS. 2-4 show a gas recognition method based on the compressive sensing theory according to an embodiment of the present disclosure, in which:
FIG. 2 is a diagram illustrating a comparison of an original signal and a reconstructed signal;
FIG. 3 is a diagram illustrating a training state of a BP neural network;
FIG. 4 is a diagram illustrating training errors of qualitative recognition.

### DETAILED DESCRIPTION

To illustrate objects, technical solutions, and advantages of the present disclosure in a clearer manner, a detailed description of the present disclosure will be given, in conjunction with specific embodiments, with reference to the drawings.

The gas recognition method based on the compressive sensing theory provided by the present disclosure applies the compressive sensing technology to the propagation procedure of the array signals in the sensor network, and uses a BP neural network to train the recognition. In this way, the computation complexity is transferred from online detection to offline network training, such that the timeliness and accuracy for the online detection done by the detection system are greatly improved.

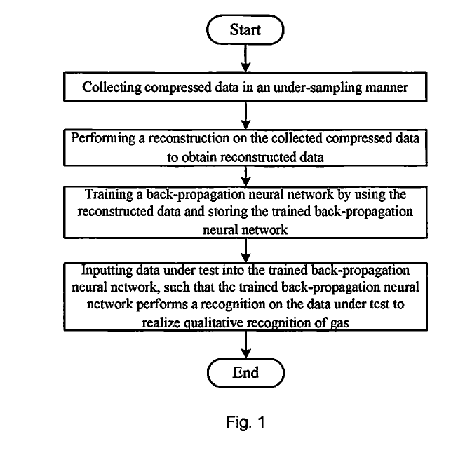

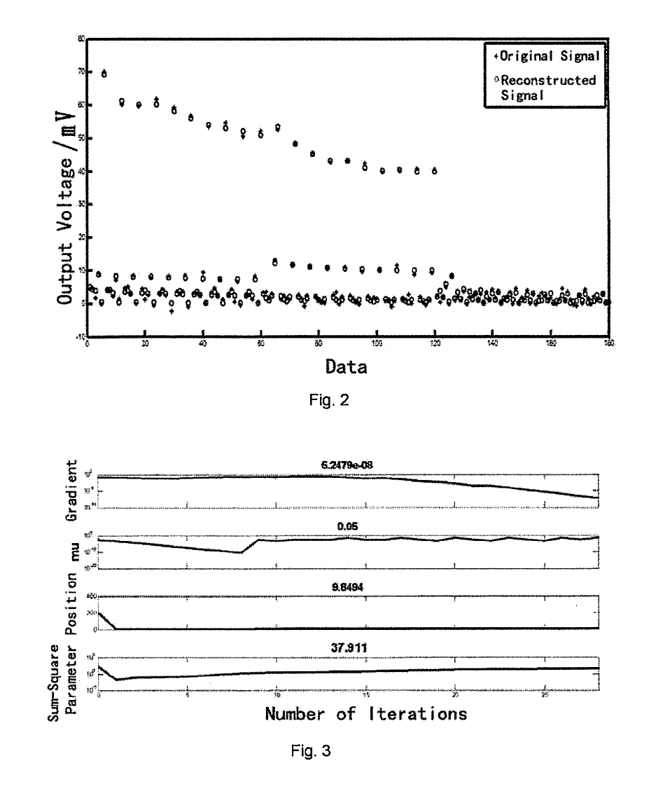

As shown in FIG. 1, it is a flow chart of a gas recognition method based on the compressive sensing theory according to an embodiment of the present disclosure. The method first collects compressed data in an under-sampling manner, and performs a reconstruction on the compressed data to obtain reconstructed data. After that, the method trains a BP network by using the reconstructed data and stores the trained network. Finally, the method inputs data under test into the trained neural network, such that the trained neural network performs recognition on the data under test to output binary quantized numbers representing different gases. In this way, a qualitative recognition of gas is achieved. In particular, the method comprises the following steps.

In step 1, compressed data is collected in an under-sampling manner.

In step 2, a reconstruction is performed on the collected compressed data to obtain reconstructed data.

In step 3, a back-propagation neural network is trained by using the reconstructed data and the trained back-propagation neural network is stored.

In step 4, data under test is input into the trained back-propagation neural network, such that the trained back-propagation neural network performs recognition on the data under test to realize qualitative recognition of gas.

In an embodiment, Step 1 of collecting compressed data in an under-sampling manner, specifically comprises: collecting compressible original data by array nodes of a sensor network; performing a sparse decomposition on the original data to acquire a first sparse matrix which is a sparse matrix correlated to the original data; performing a non-linear projection processing on the first sparse matrix to acquire a second sparse matrix whose elements are random combinations of elements of the first sparse matrix; and performing a low rate under-sampling on the data of the second sparse matrix having a greater coefficient at a frequency lower than the Nyquist sampling frequency.

In an embodiment, performing the sparse decomposition on the original data to acquire a first sparse matrix comprises: constructing a sparse matrix with a random Gaussian distribution and multiplying the collected original data by this constructed sparse matrix with the random Gaussian distribution to acquire the first sparse matrix.

In an embodiment, constructing the sparse matrix with a random Gaussian distribution comprises: selecting a Gaussian matrix with dimensions of M×N is selected, each element of which Gaussian matrix obeying the Gaussian distribution; and then normalizing each column of the Gaussian matrix to acquire a sparse matrix ψ.

In an embodiment, multiplying the collected original data by this constructed sparse matrix with the random Gaussian distribution to acquire the first sparse matrix comprises: multiplying collected compressible original data X by the constructed sparse matrix with the random Gaussian distribution ψ to acquire the first sparse matrix which is the sparsest representation of the compressible original data X, wherein if the number K of non-zero elements in the first sparse matrix is less than the number of non-zero elements in the compressible original data X, then the first sparse matrix is compressible.

In an embodiment, Step 2 of performing a reconstruction on the collected compressed data to obtain reconstructed data, comprises: selecting an observation matrix which is not correlated to the first sparse matrix; multiplying collected compressed data by the observation matrix to acquire the observed data; and performing an inverse transform on the observed data to acquire the reconstructed data. The selected observation matrix is an observation matrix P with dimensions of M×N, and the observation matrix P is not correlated to the first sparse matrix. The observed data Y is acquired by multiplying the collected compressed data X by the observation matrix P. The observed data Y is a linear combination of column vectors in the observation matrix P corresponding to the non-zero vectors in the first sparse matrix. Performing the inverse transform on the observed data is to solve X in an equation of PψX=Y, where θ=ψX. Since the number of unknown quantities in this equation set is greater than the number of equations in the equation set, the solution of X is not unique. Here, the least-1-norm is used to approximate the solution, and the final result is the reconstructed data.

In an embodiment, Step 3 of training a back-propagation neural network by using the reconstructed data and storing the trained back-propagation neural network, specifically comprises: inputting the reconstructed data, as input samples, to the back propagation neural network; iteratively processing the reconstructed data by the back propagation neural network. The iterative error in each step is compared with that in its previous step. When the iterative error reaches at an initially set threshold, the training is stopped and the back-propagation neural network is stored.

In an embodiment, Step 4 of performing a recognition by the trained back-propagation neural network on the data under test, is that: the trained back-propagation neural network compares respective connection weights during the training and outputs binary quantized numbers with the most number of similar weights, the output binary quantized numbers representing different kinds of gases, such that a qualitative recognition of gas is achieved.

Based on the flow chart of the gas recognition method based on the compressive sensing theory provided by the present disclosure as shown in FIG. 1, FIGS. 2-4 show a gas recognition method based on the compressive sensing theory according to an embodiment of the present disclosure. In particular, the method comprises the following steps.

In step 1: compressed data is collected in an under-sampling manner.

In an embodiment, a sensor array is used as a node of a sensor network. The output voltage measured by a node is X[n], where n=1, 2, 3, . . . , 180. In other words, a compressible original data X is obtained. A discrete Fourier transform is performed on the compressible original data X, to acquire a sparse representation result θ of the original data. Subsequently, a pseudo random Gaussian matrix is used as an observation matrix P to perform a non-linear projection operation on the sparse representation result. In other words, the two matrices are multiplied to acquire sparsely compressed observation data Y[m], where m=1, 2, 3, . . . , 30. It can be seen that the amount of data after the processing is less than 20% of the original amount of data.

In step 2, a reconstruction is performed on the collected compressed data to obtain reconstructed data.

The original signal is reconstructed by using the 30 pieces of compressed data. That is, the equation of PψX=Y (where θ=ψX) is solved in terms of X. Since the number of unknown quantities is greater than the number of equations in the equation set, the solution of X is not unique, and a non-linear approximation is required. Here, in the present disclosure, the least-1-norm is used to approximate the solution. A specific algorithm is to use the Orthogonal Matching Pursuit (OMP) algorithm for reconstructing the original signal. FIG. 2 is a diagram illustrating a comparison of the reconstructed data and the original data, the reconstructed data being reconstructed by compressively sensing the data collected by the sensor array. The approximation error is 0.87031.

In step 3, a back-propagation neural network is trained by using the reconstructed data and the trained back-propagation neural network is stored.

The reconstructed data is restored to a vector matrix of 6×30 as the input to the BP neural network, while the target vector is also defined as a 3 dimension matrix. Each target vector comprises three elements. The vector represents a certain gas, and an element value at its corresponding position is 1 while element values at other positions are 0. For example, for a vector corresponding to CO, the element value at its first position is 1, and element values at other two positions are 0, i.e. [1, 0, 0].

Based on the above considerations, the structure of the BP neural network used in the embodiment of the present disclosure is 6:7:3. In other words, the input layer has 6 inputs (the number of sensors of a node), the number of nodes in a single intermediate hidden layer is 7, and the output layer requires 3 neurons (the number of dimensions of the target vector). The task of qualitative detection may be achieved by both of a neural network with a single hidden layer and a neural network with multiple hidden layers. The only difference is that: the time for training a neural network with multiple hidden layers is much longer than that for training a neural network with a single hidden layer. In view of the practical problem of the present disclosure, a BP network with a single hidden layer is used to be trained with regard to reconstructed signals with different sparsity. In the present embodiment, a BP network with a single hidden layer is used, i.e. one input layer, one hidden layer, and one output layer.

During the training, the sample data is first divided into two groups. The group of data with odd numberings is the sample for training, and the group of data with even numberings is used to examine the practical performance of the trained network. A rapid BP algorithm is used to train the network, and the number of neurons in the hidden layer of the network is chosen to be 7. To have a good generalization capability of the network with respect to new inputs, the training function for the network is chosen to be trained which uses a Bayesian framework. It is assumed that the weights and thresholds for the network is a random variable with a special distribution. Then, an estimated value is acquired by a statistics method. For the training part of the network, the training samples are divided into three parts, i.e. training, verifying, and testing. The training result of the network is evaluated by using the mean square error. The training state is shown in FIG. 3.

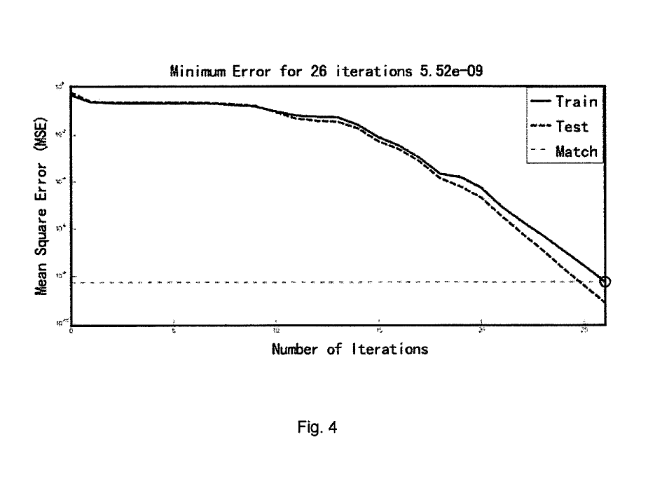

FIG. 4 is a diagram illustrating errors during the training procedure. From the figure, it can be determined that the number of iterations is 24. The object of training is achieved, and the error index converges to an expected index. The training is stopped and the trained network is stored for future usage in prediction.

Step 4: data under test is input into the trained back-propagation neural network, such that the trained back-propagation neural network performs recognition on the data under test to realize qualitative recognition of gas.

The data samples under test are input to the trained network and the input format is ensured to be same as that used in training. The trained network compares respective connection weights during the training and outputs binary quantized numbers with the most number of similar weights, the output binary quantized numbers representing different kinds of gases. The accuracy of the recognition which is output predictively by the network is 100%, and the average error of the output is only 6.0206 e⁻⁶.

In the above embodiments, X denotes the collected compressible original data, ψ denotes the sparse matrix for sparse representation, M and N denote the dimensions of the sparse matrix, θ denotes the sparse representation result of the original data, K denotes the number of non-zero elements in the sparse result or the sparsity of X representing the compressibility of the original data, P denotes the observation matrix for non-linear projection, and Y denotes the observation result of the original data.

Since, unlike the conventional method in which sampling is first performed and then redundant information is removed by compression, the compressive sensing method directly “collects” compressed information such that the amount of data being collected is reduced and the step of compression is omitted, while the advantage of simple structure of a neural network with a single hidden layer is exploited. The amount of data for transmission and storage and the accuracy of recognition are considered in a comprehensive manner, and therefore the gas recognition method based on the compressive sensing theory proposed by the present disclosure is optimal in performance.

The detailed description of the objects, solutions, and advantages of the present disclosure is given in the above specific embodiments. It is to be appreciated that the above embodiments are merely specific embodiments of the present disclosure, not for the purpose of limitations to the present disclosure. Any modification, equivalent substitution, or improvement made within the spirit and principles of the present disclosure shall be embraced by the scope of the present disclosure.

1. A gas recognition method based on the compressive sensing theory, the method comprising:
step 1 of collecting compressed data in an under-sampling manner;
step 2 of performing a reconstruction on the collected compressed data to obtain reconstructed data;
step 3 of training a back-propagation neural network by using the reconstructed data, and storing the trained back-propagation neural network; and
step 4 of inputting data under test into the trained back-propagation neural network, such that the trained back-propagation neural network performs recognition on the data under test to realize qualitative recognition of gas.

2. The gas recognition method based on the compressive sensing theory according to claim 1, wherein step 1 of collecting compressed data in an under-sampling manner, comprises:
collecting, by array nodes of a sensor network, compressible original data;
performing a sparse decomposition on the original data to acquire a first sparse matrix which is a sparse matrix correlated to the original data;
performing a non-linear projection processing on the first sparse matrix to acquire a second sparse matrix whose elements are random combinations of elements of the first sparse matrix; and
performing a low rate under-sampling at a frequency lower than the Nyquist sampling frequency on the data of the second sparse matrix having a greater coefficient.

3. The gas recognition method based on the compressive sensing theory according to claim 2, wherein performing the sparse decomposition on the original data to acquire a first sparse matrix comprises:
constructing a sparse matrix with a random Gaussian distribution and multiplying the collected original data by this constructed sparse matrix with the random Gaussian distribution to acquire the first sparse matrix.

4. The gas recognition method based on the compressive sensing theory according to claim 3, wherein constructing the sparse matrix with a random Gaussian distribution comprises:
selecting a Gaussian matrix with dimensions of M×N, each element of which Gaussian matrix obeying the Gaussian distribution; and
then normalizing each column of the Gaussian matrix to acquire a sparse matrix ψ.

5. The gas recognition method based on the compressive sensing theory according to claim 4, wherein multiplying the collected original data by this constructed sparse matrix with the random Gaussian distribution to acquire the first sparse matrix comprises:
multiplying collected compressible original data X by the constructed sparse matrix with the random Gaussian distribution ψ to acquire the first sparse matrix which is the sparsest representation of the compressible original data X,
wherein when the number K of non-zero elements in the first sparse matrix is less than the number of non-zero elements in the compressible original data X, then the first sparse matrix is compressible.

6. The gas recognition method based on the compressive sensing theory according to claim 1, wherein step 2 of performing the reconstruction on the collected compressed data to obtain reconstructed data, comprises:
selecting an observation matrix which is not correlated to the first sparse matrix;
multiplying collected compressed data by the observation matrix to acquire the observed data; and
performing an inverse transform on the observed data to acquire the reconstructed data.

7. The gas recognition method based on the compressive sensing theory according to claim 6, wherein the selected observation matrix is an observation matrix P with dimensions of M×N, and the observation matrix P is not correlated to the first sparse matrix,
wherein the collected compressed data X is multiplied by the observation matrix P, to acquire the observed data Y which is a linear combination of column vectors in the observation matrix P corresponding to non-zero vectors in the first sparse matrix.

8. The gas recognition method based on the compressive sensing theory according to claim 7, wherein the performing of an inverse transform on the observed data is to solve X in an equation of PψX=Y, where θ=ψX,
wherein since the number of unknown quantities in this equation set is greater than the number of equations in the equation set, the solution of X is not unique, and here the least-1-norm is used to approximate the solution, and the final result is the reconstructed data.

9. The gas recognition method based on the compressive sensing theory according to claim 1, wherein step 3 of training a back-propagation neural network by using the reconstructed data and storing the trained back-propagation neural network, specifically comprises:
inputting the reconstructed data, as input samples, to the back propagation neural network;
processing, by the back propagation neural network, the reconstructed data iteratively and comparing the iterative error in each step with that in its previous step; and
stopping the training and storing the back-propagation neural network when the iterative error reaches at an initially set threshold.

10. The gas recognition method based on the compressive sensing theory according to claim 1, wherein step 4 of performing, by the trained back-propagation neural network, a recognition on the data under test is that: the trained back-propagation neural network compares respective connection weights during the training and outputs binary quantized numbers with the most number of similar weights, the output binary quantized numbers representing different kinds of gases, such that a qualitative recognition of gas is achieved.
